(19) United States
(12) Patent Application Publication
(10) Pub. No.: US 2014/0273173 A1
(43) Pub. Date: Sep. 18, 2014

(54) METHOD OF SEPARATING AND RECOVERING MICROALGAE

(71) Applicant: Hirokazu Kaku, Nakano-ku (JP)

(72) Inventor: Hirokazu Kaku, Nakano-ku (JP)

(73) Assignee: KURITA WATER INDUSTRIES LTD., Tokyo (JP)

(21) Appl. No.: 14/236,395

(22) PCT Filed: Aug. 10, 2012
(86) PCT No.: PCT/JP2012/070496

§ 371 Date: May 23, 2014

(30) Foreign Application Priority Data
Aug. 12, 2011 (JP) 2011-177118
Nov. 22, 2011 (JP) 2011-254756

(51) Int. Cl. C12N 1/12

(52) U.S. Cl. CPC C12N 1/12
USPC 435/257.1

(57) ABSTRACT

Raw water containing microalgae is introduced to a pH adjusting aggregation tank, and a soluble metal salt for generating a hardly-soluble hydroxide is added for adjusting to a predetermined pH. Subsequently, microalgae is aggregated by a precipitated hardly-soluble hydroxide, and generated aggregated flocks are separated to aggregated flocks and treated water in a solid-liquid separation tank. According to the method of separating and recovering microalgae, microalgae can be recovered from a culture solution containing microalgae without using a flocculant containing aluminum or poisonous macromolecule polymer, etc. and used as a feed additive or food additive.

### TECHNICAL FIELD

The present invention relates to a method of separating and recovering efficiently separating microalgae and water of culture medium, etc. from a culture solution containing microalgae and obtaining separated water having a high microalga content, and particularly relates to a method of separating and recovering microalgae by which microalgae can be recovered to be usable as a feed additive or food additive.

### BACKGROUND ART

Microalga is a single-cell organism having a size of several μm to tens of μm. Since this microalga efficiently converts solar energy to hydrocarbon to accumulate and contains a variety of minerals and unsaturated fatty acids, etc. at a high concentration, there have been proposed a variety of utilization methods, such as using as alternative fuel of diesel fuel, etc. and as a health food typified by chlorella, etc.

As use of the microalgae as above, usages as a material for animal feed and fishery feed and as a food additive have been desired. In this regard, it is not preferable to use aluminum having particular concern for harmful influence on human body or a poisonous macromolecule polymer to aggregate microalgae for separation and recovery.

As a method of recovering microalgae as above, when it is for the purpose of producing health food or other high-value added products, it is general to use a centrifugal machine for separating and recovering microalgae in culture fluid. In this method, however, an initial investment amount for the facility is large and a power consumption is high, so that the production cost of the products becomes high, which is disadvantageous. Particularly when it is for the purpose of using the microalgae as bio fuel, there has been a problem that an energy consumption for producing surpasses energy to be obtained from produced bio fuel, so that it does not lead to a reduction of environmental burdens.

Thus, application of a technique of removing blue-green algae, which is a kind of microalgae, or red tide, etc. or a pre-treatment process in a pure water producing process may be considered. For example, as a technique of separating and removing blue-green algae, the patent document 1 discloses a technique of separating blue-green algae by performing a pressurized floatation treatment on water to be treated containing blue-green algae. Also, the patent document 2 and the patent document 3 disclose a technique of aggregating blue-green algae before a pressurized floatation treatment so as to separate blue-green algae. On the other hand, as a technique of separating and removing microalgae, a variety of methods of performing a gravity sedimentation treatment on water to be treated containing microalgae are disclosed (patent documents 4, 5 and 6). Furthermore, the patent document 7 proposes to remove blue-green algae by filtrating water to be treated containing blue-green algae with a filtration membrane, such as a microfilter and woven fabric screen.

### PRIOR ART DOCUMENTS

### Patent Documents

[Patent Document 1] Japanese Examined Patent Publication (Kokoku) No. 1991-37994

[Patent Document 2] Japanese Patent Publication (Kokai) No. 1994-315602

[Patent Document 3] Japanese Examined Patent Publication (Kokoku) No. 1990-15275

[Patent Document 4] Japanese Patent Publication (Kokai) No. 1995-289240

[Patent Document 5] Japanese Patent Publication (Kokai) No. 1995-95874

[Patent Document 6] Japanese Patent No. 2904674

[Patent Document 7] Japanese Patent Publication (Kokai) No. 2007-98342

### SUMMARY OF THE INVENTION

### Object to be Achieved by the Invention

However, in the case of separating microalgae by pressurized floatation separation without using any flocculant as described in the patent document 1, there is a problem point that an association state of microalgae is not good and a sufficient recovery rate cannot be obtained.

Therefore, as described in the patent document 2 and the patent document 3, microalgae are aggregated by using a flocculant. Due to this separation and recovery method, a recovery rate of microalgae improves and recovery performance becomes stable. As the flocculant, it is general to use an inorganic flocculant alone or to use an inorganic flocculant and a polymer flocculant together.

A method of separating and recovering microalgae by pressurized floatation separation using the flocculant may be carried out, for example, by the system shown in FIG. 3. In FIG. 3, a separation and recovery system of microalgae has the configuration that a neutralization tank 21, an aggregation tank 22 and a solid-liquid separation tank 23 having a skimmer 23A are connected by tube paths 27A and 27B serially and the skimmer 23A of the solid-liquid separation tank 23 is connected to a scum recovery tank 24, while a tube path 27C is provided at the bottom of the solid-liquid separation tank 23 and led to a treated water receiving tank 25. The treated water receiving tank 25 is connected to a pressurized water tank 26 by a drawn tube 27D and, furthermore, the pressurized water tank 26 joins with the tube path 27B via a tube path 27E. Note that, in FIG. 3, 28A and 28B indicate agitation devices, respectively, 29 a pH meter, and 30 a chemical feed pump of a NaOH aqueous solution as alkali. The chemical feed pump 30 can be controlled by a not-shown control device based on a measurement result of the pH meter 29.

In the separation and recovery system as explained above, raw water W containing microalgae is introduced to the neutralization tank 21, added with a predetermined amount of inorganic flocculant and, then, agitated rapidly by the agitation device 28A. Here, in accordance with a kind of an inorganic flocculant, pH is controlled by adding an alkali agent, such as a NaOH aqueous solution.

Subsequently, the raw water W added with the flocculant is transferred to the aggregation tank 22, furthermore agitated slowly by the agitation device 28 to form aggregation flocks of microalgae, and furthermore added with a polymer flocculant, such as polyacryl amide-based polymer, so as to coarsen the aggregation flocks.

Then, the raw water W, wherein the microalgae aggregation flocks are formed, is transferred to the solid-liquid separation tank 23, microalgae are subjected to pressurized floatation separation and gathered by the skimmer 23A, and recovered water K containing the microalgae is retained temporarily in the scum recovery tank 24, then, recovered. Thereby, microalgae can be recovered at a high concentration.

Separated water S is drawn from the bottom of the solid-liquid separation tank 23, received by the treated water receiving tank 25, supplied through the tube path 27D to the pressurized water tank 26, pressed out from the pressurized water tank 26 by an air, so that the pressurized water P from the tube path 27E joins the tube path 27B and returns to the solid-liquid separation tank 23.

However, in the separation and recovery system as explained above, it is necessary that PAC or an iron-based flocculant, etc. being typified inorganic flocculants is added in a certain amount, and aluminum derived from these inorganic flocculants and heavy metals contained as impurities are mixed in the recovered water K. Moreover, a polymer flocculant is also mixed in. Components derived from those industrial flocculants, such as PAC and iron-based flocculant, and polymer flocculants are not approved as feed additives or food additives, therefore, there is a problem that use purpose of recovered microalgae is largely limited.

On the other hand, in a method of separating microalgae by sedimentation separation as described in the patent documents 4 to 6 instead of pressurized floatation separation, specific gravity is almost equal to that of water depending on a kind of microalgae, so that sufficient separation is not attained and there is a problem that the recovery rate is low and stable treatment becomes difficult. Also, in a pre-treatment stage of solid-liquid separation, PAC, an iron-based flocculant or other industrial flocculant or a polymer flocculant is used in some cases, so that the problem explained above may not be solved thereby.

Furthermore, in the case of filtrating with a filtration membrane, such as a microfilter and woven fabric screen, as described in the patent document 7, when a woven fabric screen having an opening of several μm to tens of μm is used, there is an advantageous point that a filtration differential pressure is small and a pump power can be low, while there is a disadvantage that microalgae to be leaked increases and a recovery rate declines. On the other hand, when using a microfilter having a fine opening diameter of submicron or smaller, the recovery rate becomes high but there are problems that a filtration differential pressure is high, a power consumption is large and a flux is liable to decline due to fouling. Also, in the pre-treatment stage of solid-liquid separation, PAC, an iron-based flocculant or other industrial flocculant or a polymer flocculant is used in some cases, so that the problem explained above may not be solved thereby.

The present invention was made in consideration of the above disadvantages and has an object thereof to provide a method of separating and recovering microalgae, by which microalgae can be recovered from a culture solution containing microalgae without using a flocculant containing aluminum or a poisonous macromolecular polymer, etc. and used as a feed additive or food additive

### Means for Achieving the Object

To achieve the above object, the present invention provides a method of separating and recovering microalgae from raw water containing microalgae, characterized by comprising a hydroxide generation step for adding a soluble metal salt for generating a hardly-soluble hydroxide to raw water containing microalgae before adjusting the raw water to have pH for generating a hardly-soluble hydroxide; an aggregation step for aggregating microalgae by a precipitated hardly-soluble hydroxide; and a solid-liquid separation step for performing solid-liquid separation on aggregation flocks generated here (Invention 1).

According to the invention (Invention 1), by adding a soluble metal salt to raw water, generating a hardly-soluble hydroxide by adjusting pH, aggregating microalgae by the hardly-soluble hydroxide and forming microalgae aggregation flocks preferably, and performing solid-liquid separation on the aggregation flocks, separated water having a high microalga content can be obtained. Here, a hardly-soluble hydroxide derived from a soluble metal salt can be used as a feed additive or food additive in many cases, so that recovered microalgae can be used for those purposes.

In the invention above (Invention 1), the solid-liquid separation step is preferably pressurized floatation separation (Invention 2).

According to the invention (Invention 2), many of microalgae have specific gravity approximating to that of water, so that it takes a long time for sedimentation separation. Therefore, solid-liquid separation can be carried out efficiently by pressurized floatation separation.

In the invention above (Invention 1), as the solid-liquid separation step, screen separation can be used (Invention 3).

According to the invention (Invention 3), by aggregating microalgae by hardly-soluble hydroxide by using screen separation, solid-liquid separation can be performed efficiently.

In the inventions above (Inventions 1 to 3), the soluble metal salt is preferably usable as a feed additive or a food additive (Invention 4).

According to the invention (Invention 4), recovered microalgae can be used as a feed additive or food additive.

In the inventions above (Inventions 1 to 4), it is preferable to use discharged water from the solid-liquid separation step as a culture fluid for microalgae (Invention 5).

According to the invention (Invention 5), since discharged water of the solid-liquid separation step contains necessary nutritional components (nitride, phosphorous and minerals, etc.) for culturing microalgae, by reusing this as culture fluid for microalgae, a use amount of water and production costs of culture fluid can be reduced (cut down).

In the inventions above (Inventions 1 to 5), it is preferable to adjust discharged water from the solid-liquid separation step to have pH for generating a hardly-soluble hydroxide and to precipitate a residual soluble metal salt in the discharged water as a hardly-soluble hydroxide so as to perform solid-liquid separation (Invention 6).

According to the invention (Invention 6), a soluble metal salt can be removed from discharged water from the solid-liquid separation step, so that reuse of the discharged water from the solid-liquid separation step or discharge thereof to the external environment can become possible.

### Effects of the Invention

According to the present invention, raw water containing microalgae is added with a soluble metal salt for generating a hardly-soluble hydroxide, microalgae is aggregated by a precipitated hardly-soluble hydroxide and aggregation flocks generated here are subjected to solid-liquid separation, therefore, separated water having a high content of microalgae can be obtained. Here, by using a soluble metal salt approved as a feed additive or food additive, recovered microalgae can be used for feed or food.

### BRIEF DESCRIPTION OF DRAWINGS

[FIG. 1] A flowchart showing a system which can carry out a method of separating and recovering microalgae according to a first embodiment of the present invention.

[FIG. 2] A flowchart showing a system which can carry out a method of separating and recovering microalgae according to a second embodiment of the present invention.

[FIG. 3] A flowchart showing a system which can carry out a conventional method of separation and recovery of microalgae.

### MODE(S) FOR CARRYING OUT THE INVENTION

Below, an explanation will be made on respective embodiments of the present invention with reference to the drawings. Note that the embodiments are shown as examples and the present invention is not limited to those.

FIG. 1 shows a recovery system, which can implement a method of separating and recovering microalgae according to a first embodiment of the present invention. In FIG. 1, the system for separating and recovering microalgae has a configuration, wherein a pH adjusting aggregation tank 1 is connected with a solid-liquid separation tank 2 provided with a skimmer 2A by a tube path 6A and the skimmer 2A of the solid-liquid separation tank 2 is connected to a scum recovery tank 3, while a tube path 6B is provided to a bottom of the solid-liquid separation tank 2 and separated water S from the bottom is received by a treated water receiving tank 4. The treated water receiving tank 4 is connected with a pressurized water tank 5 by a tube path 6C and, furthermore, the pressurized water tank 5 joins a tube path 6A from a tube path 6D. Also, a lower portion of the treated water receiving tank 4 has a sloped surface and a return tube 7 is connected at the bottom.

Also, the pH adjusting aggregation tank 1 is provided with an agitation device 11, a soluble metal salt supply line 12 and a NaOH aqueous solution supply line 13 provided with a pump 13A, wherein a NaoH aqueous solution is a pH adjusting agent, while a pH meter 14 is provided in the pH adjusting aggregation tank 1. The pump 13A can be controlled by a not-shown control device based on a measurement result of the pH meter 14.

Next, an explanation will be made on a method of separating and recovering microalgae of the present embodiment using the recovery system as explained above.

(Hydroxide Generation Step)

First, after introducing raw water W containing microalgae to the pH adjusting aggregation tank 1, a soluble metal salt for generating a hardly-soluble hydroxide is added. Here, the soluble metal salt is not particularly limited as long as it can be used as a feed additive or food additive and, for example, zinc sulfate, ferrous sulfate, calcium sulfate, magnesium sulfate, manganese sulfate and potassium alum, etc. may be used.

Those soluble metal salts form hardly-soluble hydroxides when adjusted to have predetermined pH, respectively. Therefore, in the present embodiment, in order that raw water W comes to have desired pH for forming a hardly-soluble hydroxide, the pump 13A is activated based on a measurement value of the pH meter 14 to add a sodium hydroxide aqueous solution as a pH adjusting agent for adjusting the pH. As the pH adjusting agent, other than a sodium hydroxide aqueous solution, for example, a potassium hydroxide aqueous solution, sodium carbonate aqueous solution and other alkali are generally used. As a pH value to be adjusted, an appropriate value is selected in accordance with a soluble metal salt to be used. For example, pH 7 to 10 is preferable when using zinc sulfate, pH 7 to 11 is preferable when using ferrous sulfate, and pH 6 to 8.5 is preferable when using potassium alum. Note that when the pH is already in a preferable range in a state where a soluble metal salt is added to the raw water W, addition of a pH adjusting agent may be omitted.

By forming an insoluble metal hydroxide by adjusting pH when needed as explained above and agitating by the agitation device 11, Zn²⁺, Fe²⁺, Ca²⁺ or other cation in the raw water W neutralizes surface charges of microalgae, and flocks are formed due to a coagulation effect. Furthermore, Zn(OH)₂, Fe(OH)₂ or other hardly-soluble metal hydroxide is formed from the cation to progress formation of flocks.

An amount of a soluble metal salt to be added may be selected arbitrarily in accordance with a microalga concentration (SS concentration) in raw water (water to be treated) W, however, it is normally selected from a range of several to hundreds of mg/L for the purpose of suppressing a mixed amount in recovered microalgae as much as possible, and a minimum necessary concentration is preferable.

As explained above, by adjusting pH in accordance with need to form an insoluble metal hydroxide and agitating by the agitation device 11, neutralization of charges of suspended particles and formation of flocks progress furthermore. Retention time in the pH adjusting aggregation tank 1 for performing aggregation step is preferably 5 to 10 minutes, and an agitation speed by the agitation device 11 is preferably 0.3 to 3 m/sec. as a rim speed.

(Solid-Liquid Separation Step)

Subsequently, after generating aggregation flocks in the aggregation step above, the aggregation flocks and treated water are separated in the solid-liquid separation tank 2. Specifically, microalgae are separated by pressurized floatation and gathered by a skimmer 2A, and recovered water K containing the microalgae is retained temporarily in the scum recovery tank 3, then, recovered. As a result, microalgae can be recovered at a high concentration.

On the other hand, since microalgae is also contained in the separate water S in the solid-liquid separation tank 2, the separated water S is drawn from a lower portion of the solid-liquid separation tank 2 and received by the treated water receiving tank 4. In the present embodiment, by forming a bottom of the treated water receiving tank 4 to be a concave slope, microalgae settled by gravity are gathered and returned as return water B from the tube 7 to raw water W. Here, since zinc and iron, etc. added as soluble metal salts are mineral elements necessary for growing microalgae, there is no problem when reusing discharged water from the treated water receiving tank 4 as culture fluid for microalgae, moreover, it is possible to obtain the effect of decreasing water to be used and an amount of nutrients.

Return of microalgae from the return tube 7 to raw water W as explained above is preferably performed so as to raise a microalgae concentration in the raw water W to be 10 to 200 mg/L, particularly 30 to 80 mg/L. When raise of the microalgae concentration in the raw water W by returning is less than 10 mg/L, the effect of improving a recovery rate by the returning is not sufficient, while when exceeding 200 mg/L, an amount of a soluble metal salt required for forming flocks increases, which may results in an increase of residual soluble metal salt in recovered water K, which is not preferable.

On the other hand, since microalgae contained in the separated water S left in the treated water receiving tank 4 floats to be separated easily, further flock formation is not necessary, so that it is supplied to the pressurized water tank 5 by the tube path 6C and pushed out from the pressurized water tank 5 by an air, so that pressurized water P from the tube path 6D joins the tube path 6A and returns to the solid liquid separation tank 2.

From the respective steps as explained above, after adding a soluble metal salt to raw water W to adjust the pH, by recovering microalgae and returning non-recovered microalgae to circulate for recovering, an adding amount of the soluble metal salt can be reduced largely and microalgae can be effectively recovered at a high recovery rate. Moreover, recovered microalgae may be usable not only as fuel but also usable as feed, food or additives thereto.

Furthermore, according to the first embodiment as explained above, neutralization and aggregation are carried out in a single tank of the pH adjusting aggregation tank 1, therefore, the number of tanks is small and the apparatus can be simplified and, moreover, an adding amount of a soluble metal salt can be decreased, so that there is an effect that a necessary amount of NaOH to be used for pH adjustment can be also reduced. As a result, in a condensation step and drying step as post-treatment steps performed later in accordance with need, a scale of apparatuses and facility may be reduced, and costs and energy consumption in those steps can be reduced.

Next, a second embodiment will be explained in detail based on FIG. 2. In FIG. 2, same reference numbers are added to the same components as those in the first embodiment explained above and detailed explanation thereof will be omitted.

In the second embodiment, as a means to separate aggregation flocks and treated water in the solid-liquid separation tank 2, a screen 2B is provided instead of the skimmer 2A, and the same configuration and function effects as those in the first embodiment explained above are given except for not returning the separated water S. By using a screen 2B as the recovery means in this way and filtrating microalgae on the screen 2B, recovery can be attained effectively.

As above, the present invention was explained with reference to the attached drawings, however, the present invention is not limited to the first and second embodiments explained above and may be implemented in variously modified ways. For example, the present invention is characterized by adding a soluble metal salt for generating a hardly-soluble hydroxide to raw water W, before aggregating microalgae by the pH adjusted and precipitated hardly soluble hydroxide. Accordingly, in the first embodiment, although pressurized floatation separation using the solid-liquid separation tank 2 was performed as a solid-liquid separation method, the solid-liquid separation step is not particularly limited and, for example, a gravity sedimentation method, centrifugal sedimentation method or other sedimentation method, a low-pressure floatation method or other floatation method, etc. may be selected arbitrarily and used in accordance with a property of aggregation flocks.

Furthermore, when disposing of the treated water S, cation derived from a soluble metal salt is again adjusted to have a pH for generating a soluble hydroxide, residual soluble metal salt in the treated water S is brought to be precipitated as hardly-soluble hydroxide and subjected to solid-liquid separation together with residual hardly-soluble metal salt in the treated water S, as a result, a treatment system conforming to environmental regulations can be attained.

### EXAMPLES

Below, the present invention will be explained further in detail based on examples and comparative examples below, however, the present invention is not limited to the examples below.

### Example 1

As microalgae, raw chlorella “V12” (produced by Chlorella Industry Co., Ltd) was added to municipal water to have an SS (chlorella) concentration of 400 to 500 mg/L, so that raw water W was prepared.

A recovery test of microalgae was conducted by using a pilot test device having the configuration shown in FIG. 1 and setting a treatment amount of the raw water W to be 100 L/hour. A capacity of the pH adjusting aggregation tank 1 was 10 L, retention time was 6 minutes, agitation rim speed was 1 m/sec., zinc sulfate heptahydrate (produced by Wako Pure Chemical Industries, Ltd.: CAS No. 7733-02-0) was added as a soluble metal salt to the pH adjusting aggregation tank 1 at a rate of 1 to 50 g/hour, while a sodium hydroxide aqueous solution was added from the NaOH aqueous solution supply line 13 to adjust the pH to be 9.0, and an insoluble metal hydroxide and aggregation flocks of microalgae were formed.

Next, raw water W, wherein aggregation flocks are generated, is introduced to the solid-liquid separation tank 2 having a capacity of 13 L, solid-liquid separation was performed for a retention time of 8 minutes, and recovery water K was recovered. Separated water S of the solid-liquid separation tank 2 was retained temporarily in the treated water receiving tank 4 having a capacity of 5 L before being discharged. A part of the separated water S was returned as a pressurized water through the pressurized water tank 5 to the solid-liquid separation tank 2 via the tube path 6D.

An adding concentration of zinc sulfate heptahydrate was optimized to be a concentration in the above range, with which a recovery rate (R) of microalgae is high and an adding amount can be minimum. Then, a microalgae concentration (C1) and a flow amount (Q1) of the raw water W and a microalgae concentration (C2) and flow amount (Q2) of floatation separation treated water were measured, respectively, and a recovery rate (R) of microalgae was calculated based on the formula below.

R(%)=[1−(C2×Q2)+(C1×Q1)]×100

Also, water was sampled at a pH adjusting aggregation tank outlet 1 and diameters of aggregated microalgae flocks were measured. The results are shown along with a scum surfacing speed and recovered microalgae concentration in Table 1.

### Example 2

Other than adding ferrous sulfate heptahydrate (produced by Wako Pure Chemical Industries, Ltd.: CAS No. 7782-63-0) as a soluble metal salt at a rate of 1 to 50 g/hour to the pH adjusting aggregation tank 1, a test was conducted in the same way as in the example 1. The result is also shown in Table 1.

### Comparative Example 1

Other than using the apparatus shown in FIG. 3, adding an anion-based polymer flocculant (“Kuriflock PA331” produced by Kurita Water Industries Ltd.) at a rate of 50 to 200 mg/hour, adding polyaluminum chloride at 5 to 50 g/hour and adding a sodium hydroxide aqueous solution to adjust the pH to 6.5, a test was conducted in the same way as in the example 1. Note that the retention time in the aggregation tank 22 was 3 minutes and agitation rim speed was 0.8 m/sec.

TABLE 1

| | | | Comparative |
|---|---|---|---|
| | Example 1 | Example 2 | Example 1 |
| Aggregated Flock | 0.2 to 0.1 | 0.8 to 2.3 | 0.5 to 10.0 |
| Diameter (mm) | | | |
| Scum Surfacing | 36 to 72 | 36 to 48 | 21 to 48 |
| Speed (m/hour) | | | |
| Microalgae | 84.3 to 99.5 | 94.3 to | 81.3 to |
| Recovery Rate | | 97.0 | 97.9 |
| (Average Value) | | | |
| (%) | | | |
| Recovered | 1.3 to 5.0 | 2.5 | 2.5 to 5.0 |
| Microalgae | | | |
| Concentration | | | |
| (Average Value) | | | |
| (wt %) | | | |

### Examples 3 and 4 and Comparative Example 2

Other than using raw water W prepared from microalgae material obtained by growing Scenedesmus “NIES-96 plants” obtained from Microbial Culture Collection at National Institute for Environmental Studies as microalgae on C culture medium shown in Table 2 and Table 3 added with water so as to attain an SS (Scenedesmus) concentration of 400 to 500 mg/L, a test was conducted in the same way as in the examples 1 and 2 and comparative example 1. The results are shown in Table 4.

TABLE 2
C Culture Medium Composition

| Composition | Amount | |
|---|---|---|
| Ca(NO₃)₂•4H₂O | 15 | mg |
| KNO₃ | 10 | mg |
| β-Na2glycerophosphate•5H₂O | 5 | mg |
| MgSO₄•7H₂O | 4 | mg |
| Vitamin B₁₂ | 0.01 | μm |
| Biotin | 0.01 | μm |
| Thiamine HCl | 1 | μm |
| PIV metals* | 0.3 | mL |
| Tris(hydroxymethyl)aminomethane | 50 | mg |
| Distilled Water | 99.7 | mg |

pH 7.5
*Composition shown in Table 3

TABLE 3
PIV metal Composition

| Composition | Amount | |
|---|---|---|
| NaEDTA•2H₂O | 100 | mg |
| FeCl₃•6H₂O | 19.6 | mg |
| MnCl₂•4H₂O | 3.6 | mg |
| ZnCl₂ | 1.04 | mg |
| CoCl₂•6H₂O | 0.4 | mg |
| Na₂MoO₄•2H₂O | 0.25 | mg |
| Distilled Water | 100 | mL |

TABLE 4

| | | | Comparative |
|---|---|---|---|
| | Example 3 | Example 4 | Example 2 |
| Aggregated | 0.5 to 1.5 | 0.2 to 0.8 | 0.8 to 7.0 |
| Flock Diameter | | | |
| (mm) | | | |
| Scum Surfacing | 36 to 48 | 21 to 48 | 48 to 72 |
| Speed (m/hour) | | | |
| Microalgae | 93.7 to 98.4 | 81.4 to 98.2 | 96.2 to 97.7 |
| Recovery Rate | | | |
| (Average Value) | | | |
| (%) | | | |
| Recovered | 2.5 | 1.3 to 2.5 | 2.5 to 3.3 |
| Microalgae | | | |
| Concentration | | | |
| (Average Value) | | | |
| (wt %) | | | |

As is clear from Table 1 and Table 4, comparing with the comparative examples 1 and 2, wherein an anion-based polymer flocculant and polyaluminum chloride used in conventional effluent treatment are used, in the examples 1 and 2 and examples 3 and 4, wherein a soluble metal salt for forming a hardly-soluble hydroxide was used, diameters of aggregation flocks of microalgae were small, however, equivalent or better performances were exhibited in the scum surfacing speed and microalgae recovery rate, accordingly, it was confirmed that there was no problem in terms of practical use. Note that recovered microalgae were usable for feed and food.

### INDUSTRIAL APPLICABILITY

According to the method of separating and recovering microalgae of the present invention as explained above, since a soluble metal salt for generating a hardly-soluble hydroxide is added for aggregation of microalgae, microalgae are aggregated by the hardly-soluble hydroxide derived from the soluble metal salt, and aggregation flocks generated here are subjected to solid-liquid separation, separated water having a high microalga content can be obtained. Here, since the hardly-soluble hydroxide derived from a soluble metal salt can be used as a feed additive or food additive in many cases, recovered microalgae can be used for those purposes.

### EXPLANATION OF REFERENCE NUMBERS

1 . . . pH adjusting aggregation tank (adjustment step, aggregation step)
2 . . . solid-liquid separation tank (solid-liquid separation step)
2A . . . skimmer (solid-liquid separation step)
3 . . . scum recovery tank (solid-liquid separation step)
7 . . . return tube (return step)
12 . . . soluble metal salt supply line
13 . . . NaOH aqueous solution supply line (adjustment step)
W . . . raw water
S . . . separated water
K . . . recovered water
B . . . return water

1. A method of separating and recovering microalgae from raw water containing microalgae, comprising:
a hydroxide generation step for adding a soluble metal salt for generating a hardly-soluble hydroxide to raw water containing microalgae before adjusting the raw water to have pH for generating a hardly-soluble hydroxide;
an aggregation step for aggregating microalgae by a precipitated hardly-soluble hydroxide; and
a solid-liquid separation step for performing solid-liquid separation on aggregation flocks generated here.

2. The method of recovering microalgae as set forth in claim 1, wherein the solid-liquid separation step is pressurized floatation separation.

3. The method of recovering microalgae as set forth in claim 1, wherein the solid-liquid separation step is screen separation.

4. The method of recovering microalgae as set forth in claim 1, wherein the soluble metal salt may be used as a feed additive or a food additive.

5. The method of recovering microalgae as set forth in claim 1, wherein reusing discharged water from the solid-liquid separation step as a culture fluid for microalgae.

6. The method of recovering microalgae as set forth in claim 1, wherein adjusting discharged water from the solid-liquid separation step to have pH for generating a hardly-soluble hydroxide, and precipitating a residual soluble metal salt in the discharged water as a hardly-soluble hydroxide so as to perform solid-liquid separation.

7. The method of recovering microalgae as set forth in claim 2, wherein the soluble metal salt may be used as a feed additive or a food additive.

8. The method of recovering microalgae as set forth in claim 2, wherein reusing discharged water from the solid-liquid separation step as a culture fluid for microalgae.

9. The method of recovering microalgae as set forth in claim 2, wherein adjusting discharged water from the solid-liquid separation step to have pH for generating a hardly-soluble hydroxide, and precipitating a residual soluble metal salt in the discharged water as a hardly-soluble hydroxide so as to perform solid-liquid separation.

10. The method of recovering microalgae as set forth in claim 3, wherein the soluble metal salt may be used as a feed additive or a food additive.

11. The method of recovering microalgae as set forth in claim 3, wherein reusing discharged water from the solid-liquid separation step as a culture fluid for microalgae.

12. The method of recovering microalgae as set forth in claim 3, wherein adjusting discharged water from the solid-liquid separation step to have pH for generating a hardly-soluble hydroxide, and precipitating a residual soluble metal salt in the discharged water as a hardly-soluble hydroxide so as to perform solid-liquid separation.

13. The method of recovering microalgae as set forth in claim 4, wherein reusing discharged water from the solid-liquid separation step as a culture fluid for microalgae.

14. The method of recovering microalgae as set forth in claim 4, wherein adjusting discharged water from the solid-liquid separation step to have pH for generating a hardly-soluble hydroxide, and precipitating a residual soluble metal salt in the discharged water as a hardly-soluble hydroxide so as to perform solid-liquid separation.

15. The method of recovering microalgae as set forth in claim 5, wherein adjusting discharged water from the solid-liquid separation step to have pH for generating a hardly-soluble hydroxide, and precipitating a residual soluble metal salt in the discharged water as a hardly-soluble hydroxide so as to perform solid-liquid separation.
